(19) United States
(12) Patent Application Publication
(10) Pub. No.: US 2025/0168221 A1
(43) Pub. Date: May 22, 2025

(54) CONNECTIVITY TOLERANT MEDICAL INFORMATION SYSTEM AND METHOD

(71) Applicant: Halo Tech Corp., Portola Valley, CA (US)

(72) Inventors: Aram Compeau, Salem, NH (US); Aleksandar Kolev, Sofia (BG); Kiril Nedialkov, Sofia (BG); Ilia Vatahov, Sofia (BG); Aleksandar Gudev, Sofia (BG)

(21) Appl. No.: 18/517,265

(22) Filed: Nov. 22, 2023

(51) Int. Cl. H04L 67/10; G06F 3/06; G06F 16/174; G06F 16/178; G16H 10/60; G16H 30/20

(52) U.S. Cl. CPC H04L 67/10; G06F 3/0608; G06F 3/0641; G06F 3/0661; G06F 3/067; G06F 16/1744; G06F 16/1752; G06F 16/1794; G16H 10/60; G16H 30/20

(57) ABSTRACT

A system, method, and device for providing a service to a set of client systems is disclosed. The method includes (i) connecting, by one or more processors associated with an edge system, to a cloud service, (ii) maintaining an edge index of data stored locally at the edge system, (iii) syncing, based on the edge index, at least a subset of the data comprised at the edge system with cloud data stored at the cloud service, and (iv) providing access to the data stored locally at the edge system to one or more client systems connected to the edge system.

### BACKGROUND OF THE INVENTION

The medical field heavily relies on accurate and timely access to medical imaging data for diagnosing various conditions and devising appropriate treatment strategies. Medical practitioners, such as radiologists, physicians, and surgeons, require a robust and user-friendly platform that allows them to analyze medical images efficiently and communicate with their peers effectively.

Existing PACS systems have facilitated medical image storage and retrieval but are often limited in providing advanced image analysis tools and comprehensive service offerings. For example, existing systems have problems delivering quality patient care in the face of connectivity disruptions.

### BRIEF DESCRIPTION OF THE DRAWINGS

Various embodiments of the invention are disclosed in the following detailed description and the accompanying drawings.

FIG. 1A is a block diagram of a network system according to various embodiments of the present application.

FIG. 1B is a block diagram illustrating an embodiment of a local data store.

FIG. 1C is a block diagram illustrating an embodiment of a cloud data layer.

FIG. 2 is a block diagram of an edge system for providing service to client devices according to various embodiments of the present application.

FIG. 3 is a block diagram of a cloud system for providing service to client devices via an edge service according to various embodiments of the present application.

FIG. 4 is a flow diagram of a method for providing access to data at an edge system according to various embodiments of the present application.

FIG. 5 is a flow diagram of a method for managing data at a cloud system for use by an edge service according to various embodiments of the present application.

FIG. 6 is a flow diagram of a method for transferring data between an edge system and a cloud system according to various embodiments of the present application.

FIG. 7 is a flow diagram of a method for storing data according to various embodiments.

FIG. 8 is a flow diagram of a method for prefetching data to be stored at the edge system in advance of an event according to various embodiments.

FIG. 9 is a flow diagram of a method for providing service at an edge system based on a connection between the edge system and the cloud system according to various embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

As used herein, Digital Imaging and Communications in Medicine (DICOM) means a standard that specifies a non-proprietary data interchange protocol, digital image format, and file structure for biomedical images and image-related information.

As used herein, a Picture Archiving and Communication System (PACS) server is a computing device for securely storing, retrieving, managing, and accessing medical imaging information. For example, a PACS server is a centralized device allowing data access requests to multiple other devices. In related art systems, PACS servers are sometimes restricted to providing services to other devices on a same local area network (LAN). In various embodiments, a local PACS server is instantiated at the edge service to provide PACS server functionality to client systems (e.g., computers or medical image systems) that connect to the edge service. In some embodiments, a local PCS server includes the capability to securely store, retrieve, manage, and access medical imaging data and is deployed locally in a center (e.g., a local radiology center) or at a remote location (e.g., a home system). A physical PACS server may be comprised at the cloud service to securely store/persist the entire medical record dataset for a tenant (e.g., a medical practice).

Related art systems (e.g., providing the services such as scheduling medical procedures, performing medical imaging, or enabling medical record assessment by a medical professional) generally fall into two distinct categories: (a) fully software as a service (SaaS) services, or (b) fully on premises deployments. Neither category of related art systems provides a resilient service in the face of connectivity disruptions. Various embodiments provide a service that is more resilient to temporary loss of connectivity. For example, various embodiments comprise a SaaS platform (e.g., a cloud service) and an on-premises platform (e.g., the edge service), which provides a client system with some functionality in the face of connectivity disruptions.

In some embodiments, the cloud service provides a blob storage in which various studies (e.g., medical images, medical charts, etc.) or other medical data is stored. The cloud service may collect medical data from various edge services or client systems, such as various medical imaging systems associated with a particular tenant for which the cloud service provides services. Examples of medical data include medical images, medical requisition information, patient studies, lab reports, or patient biographical information.

In some embodiments, the edge service (e.g., an on-premises deployment) comprises a local storage to provide access/service to client systems in the face of connectivity disruptions with the cloud service. The edge service stores at the local storage newly generated medical data (e.g., newly acquired medical images from a connected medical imaging system) and medical data pre-fetched (e.g., using an image transfer component, such as a transfer agent) from the cloud service. The pre-fetched medical data may correspond to medical data (e.g., studies, images, medical records, etc.) that is expected to be used at a client system (e.g., within a predetermined time period, within a threshold period of time, or within a predetermined number of medical professional assessments, etc.). In response to determining that a connection between the cloud service and the edge system is lost, the one or more client systems use the data stored locally at the edge service (e.g., the edge system), and new data generated by the one or more client systems is stored locally at the edge service until the connection is restored between the cloud service and the edge system. In response to determining that the connection is restored, at least a subset of the new data stored locally at the edge service is synced with the cloud service. In some embodiments, syncing data between the edge service and the cloud service includes translating edge data stored locally at the edge system to a predefined data format associated with the cloud service, and causing translated edge data to be transferred to the cloud service.

In some embodiments, the edge service locally deploys third party software that is compatible with the cloud service and/or connected client systems (e.g., medical imaging systems, computers used by medical professionals, etc.). The edge service (e.g., the locally deployed third-party software) allows for the base functionality (e.g., a subset of the full suite of services provided by the cloud service, such as scheduling, image collection, medical record review/annotation, etc.) and local storage of studies (e.g., to locally capture images).

Whenever there is a radiologist that needs to read a medical record (e.g., a study, such as a medical image captured by a medical imaging system), the cloud initiates a transfer to the applicable edge component. The data to be used by the radiologist (e.g., the data expected to be required in analyzing the medical record) becomes available at the edge component. For example, the edge service provides a local PACS server that creates the illusion of an on-premises/local PACS server. In some embodiments, the local PCS server has two components 1) a local data store that is a file system with two components: directed acyclic graph (DAG) storage—where artifacts are stored and block storage—where the compressed blocks are stored with each block representing a collection of chunks; and 2) a DICOM communication or comms layer—which speaks the DICOM language that all scanners and viewers speak. The edge service may asynchronously pre-fetch the data expected to be used by the radiologist.

Both DICOM and PACS were designed for LAN access and not designed/configured for internet access or the deployment as a cloud service. Accordingly, various embodiments include a PACS server deployed at the cloud service and a local PACS server deployed at the edge service. The instantiation/deployment of the local PACS server enables third-party applications configured to store data (e.g., DICOM format files) to a PACS server to interact with the local PACS server (e.g., thereby replicating the LAN access to the PACS server used in related art systems). The protocols for related art systems are not configured for a wide area network—for example, such protocols lack encryption and push data through PACS systems file-by-file, which is extremely inefficient (e.g., each scan may comprise thousands of files). Various embodiments thus enable conventional medical imaging systems or medical reading systems to communicate according to the industry standard DICOM and PACS protocols (e.g., even in the face of connection disruptions) while leveraging the benefits of a cloud service, such as more compute resources for the deployment/use of machine learning models. Typical medical device systems use the inefficient DICOM protocol to send and receive data from/to a local PACS system. In some embodiments, the system's local PACS uses different protocols to efficiently send and retrieve imaging data to and from a cloud-based system.

Various embodiments provide a system, method, and device for providing service to a set of client systems. The system comprises an edge system comprising one or more processors, and a memory coupled to the one or more processors and configured to provide the one or more processors with instructions. The one or more processors of the edge system are configured to: (i) connect to a cloud service, (ii) maintain an edge index of data stored locally at the edge system, (iii) sync, based on the edge index, at least a subset of data comprised at the edge system and the cloud service, and (iv) provide access to the data stored locally at the edge system to one or more client systems connected to the edge system. In some embodiments, the edge index comprises a study index, which indexes the data that is stored in the Data Store component. The study index maps each resource (snapshot, saved in Data Store) to a patient, to an exam, etc. In some embodiments, the data store contains two components: 1) a DAG store and 2) a block store, but they are hidden from the rest of the system. In some embodiments, the data store provides a simple interface that enables the following functionality: 1) creating a snapshot from raw data; 2) getting a snapshot using a snapshot identifier; and 3) getting a DAG for some snapshot (used by Transfer Component). In some embodiments, the data store works like a file system. For example, the study index can be asked for the latest study for particular patient, and in response the requester will receive a snapshot identifier (e.g., zgW9uWDFY9HZPgiYh9QcyWH4kQvNjMALGgaqHd9xpQRBBv5), which can then be used to access the actual data from the data store. In some embodiments, the data store will use the snapshot identifier to extract the DAG and decompress the blocks.

In some embodiments, the data store comprises two indices: a block index (e.g., data index or medical data index) and a DAG index (e.g., artifact index). In some embodiments, the block index comprises data that maps each resource (e.g., medical data, medical study, medical report, etc.) to a patient, to an exam, to an appointment, to an identifier, to a doctor, or any other appropriate other item. The block index is used also for making decisions about what data is to be synced between the edge and the cloud. For example, if the system or user wants to send all local studies for the past two hours, the block index is queried to give us the snapshot identifiers of those studies, then the system will use a transfer module or agent to send the actual data of the local studies. In some embodiments, the transfer module or agent uses the local data store and its block index to determine what chunks have to be sent, retrieve the blocks that contain those chunks, and transmit those blocks to the cloud. In some embodiments, the DAG index is used as a mechanism for identifying chunks and/or blocks that contain the chunks that have been previously stored or transmitted and stored so that deduplication of the chunks and/or blocks can be used to make storage and transmission between the edge system and the cloud system more efficient.

Various embodiments provide a system, method, and device for providing service to a set of client systems. The system comprises a cloud system to provide the cloud service to an edge component. The one or more processors of the cloud system are configured to (a) connect to the edge system, (b) maintain a cloud index of data stored locally at the cloud system, and (c) sync, based on the cloud index, at least the subset of data comprised at the edge system and the cloud service.

In some embodiments, syncing the at least the subset of data comprised at the edge system and the cloud system comprises: determining the subset of data to be transferred between the edge system and the cloud system based at least in part on the edge index and the cloud index. In some embodiments, determining the subset of data to be transferred between the edge system and the cloud system comprises determining cloud data to be transferred from the cloud system to the edge system, which may include (a) determining a set of cloud files to be transferred to the edge system, (b) determining a plurality of chunks corresponding to the set of cloud files to be transferred, (c) determining, based at least in part the edge index, a subset of cloud chunks of the plurality of cloud chunks to transfer to the edge device based on a determination of which of the plurality of cloud chunks the edge device stores copies as edge chunks, and (d) causing the subset of cloud chunks to be transferred to the edge system. In some embodiments, determining the subset of data to be transferred from the cloud system to the edge system: (a) determining the snapshot identifier (e.g., a snapshotId, a hash of the data, etc.) for the data that is to be sent (e.g., medical artifact, where each artifact is a collection of files by using the medical data index; (b) In the transfer module (cloud)—using the snapshot identifier to extract the DAG, which contains the hashes of each chunk of the data to be sent, from the cloud data store and send it to the edge; (c) In the edge (using the edge's data store) compare the received DAG for the incoming snapshot with the DAGs of all locally available snapshots to determine chunks already stored at the edge and the missing chunks not stored at the edge; and (d) (edge) download to the edge system from the cloud system all blocks that contain the missing chunks and store in the edge index the location of each of the blocks/chunks and their identifiers to be able to reconstruct the transferred data.

In some embodiments, the causing of the subset of the cloud chunks to be transferred to the edge system comprises grouping the subset of the cloud chunks into a group of blocks, wherein a first block (and actually all subsequent blocks as well) of the group of blocks comprises a plurality of chunks comprised in the subset of the cloud chunks and transferring the group of blocks to the edge system. In some embodiments, the subset of cloud chunks is grouped into blocks and the blocks are then compressed (e.g., the blocks are each compressed in a similar manner). The subset of cloud chunks is transferred based on a deduplication of edge chunks stored at the edge system. In some embodiments, the data in a block is compressed upon creation of the block. In some embodiments, after performing a deduplication, the cloud system/service performs a compression on a deduplicated set of cloud chunks. The deduplication of edge chunks is determined based at least in part on a hash or signature associated with each edge chunk or cloud chunk.

The system improves a computer by providing software to enable interconnection of systems in distributed locations (e.g., medical imaging systems) by utilizing a cloud-based service. The cloud-based service enables efficient storage and access to data (e.g., medical data) by translating standard formats (e.g., medical standard formats—such as DICOM protocol files) and chunking data as well as deduplicating and compressing to aid in transferring and storing data. In addition, the system manages synchronizing the storage of data between distributed locations or edge systems as well as appropriately moving data to locations where it is needed (e.g., moved to a location where a medical professional needs to be able to access the data).

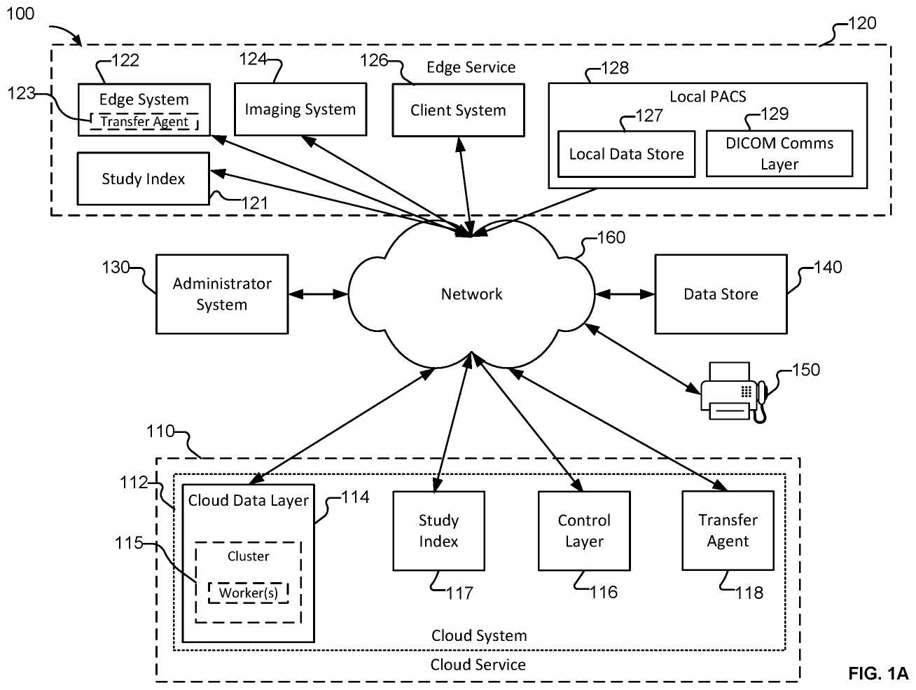

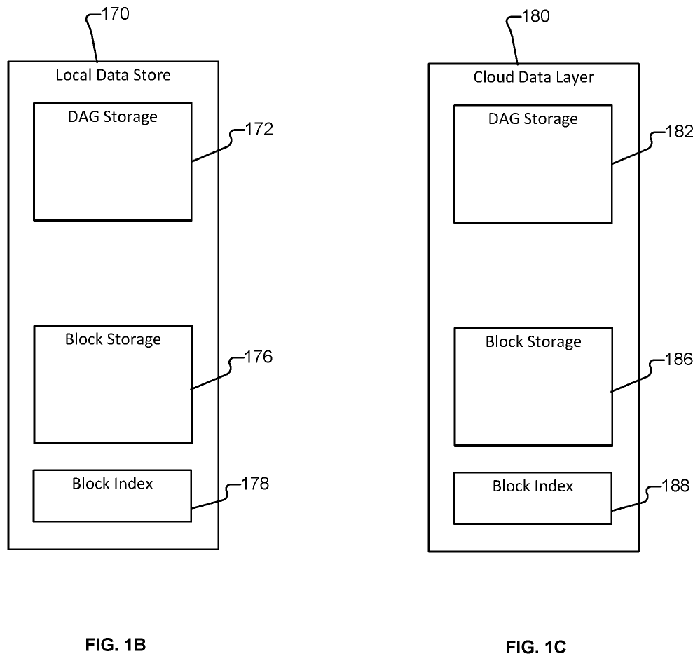

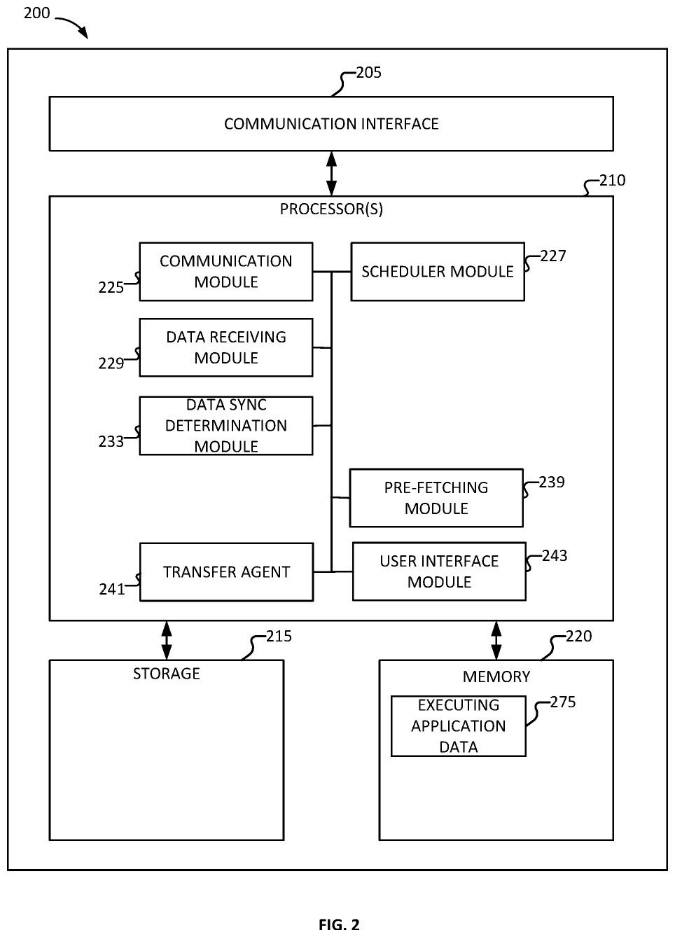

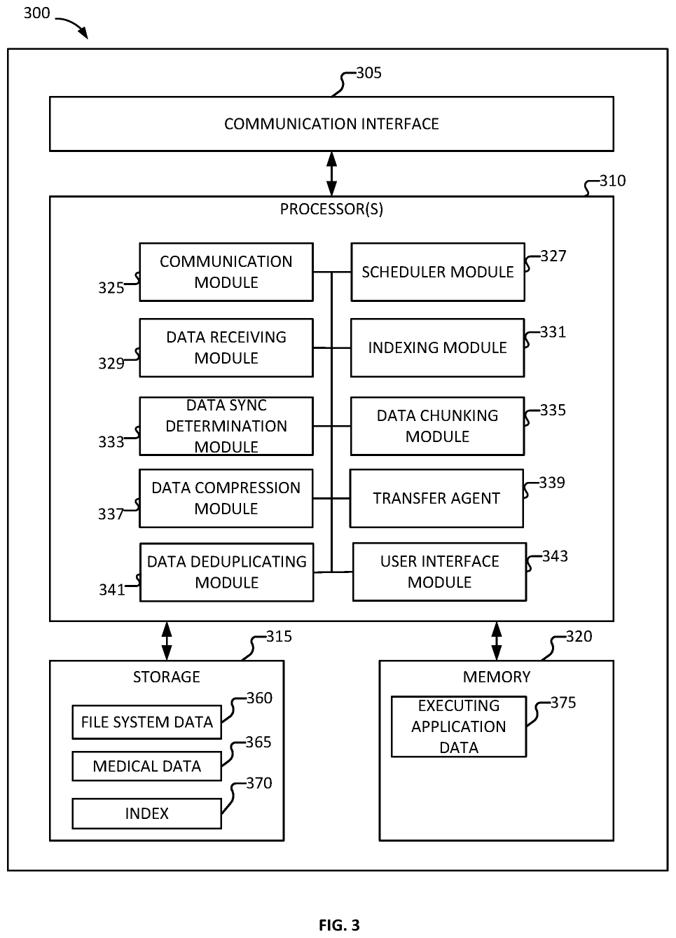

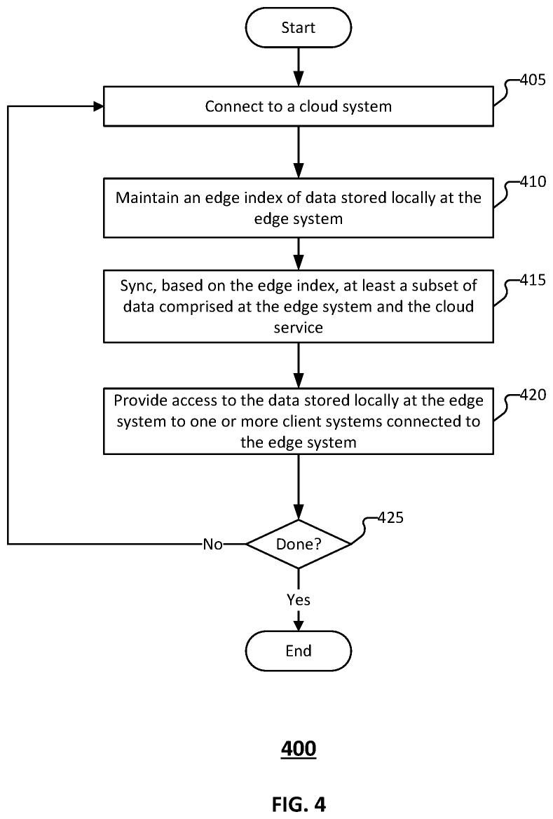

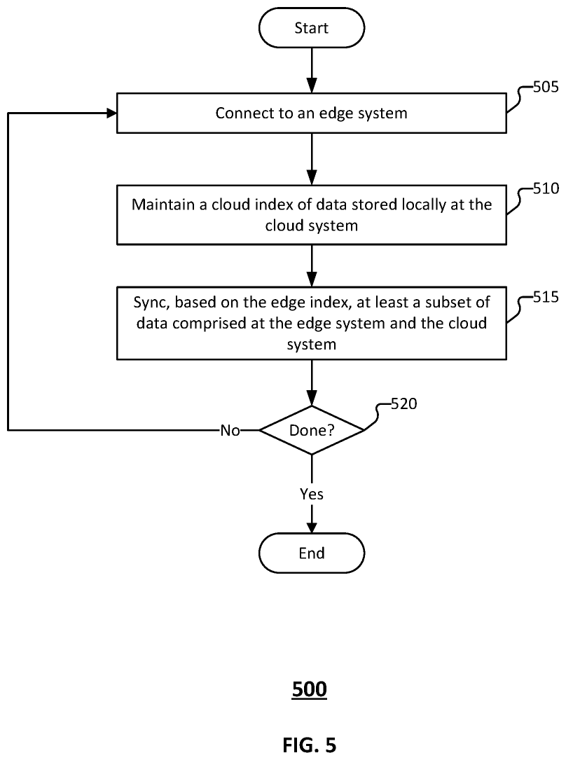

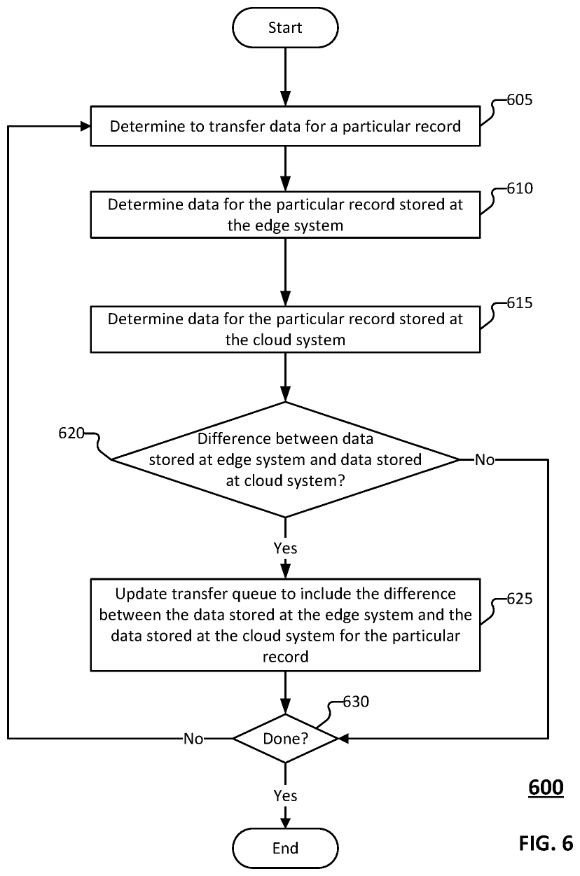

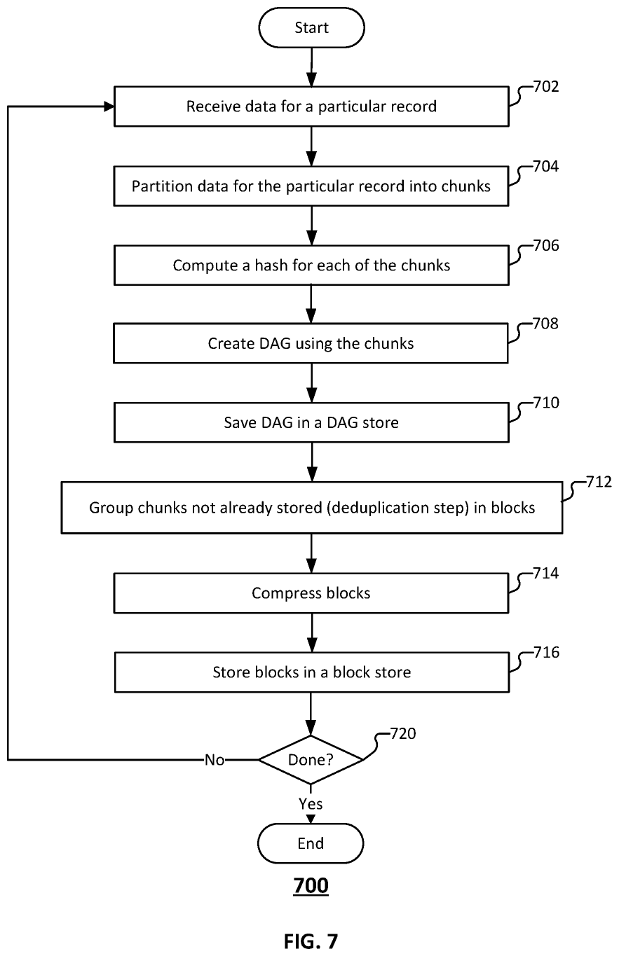

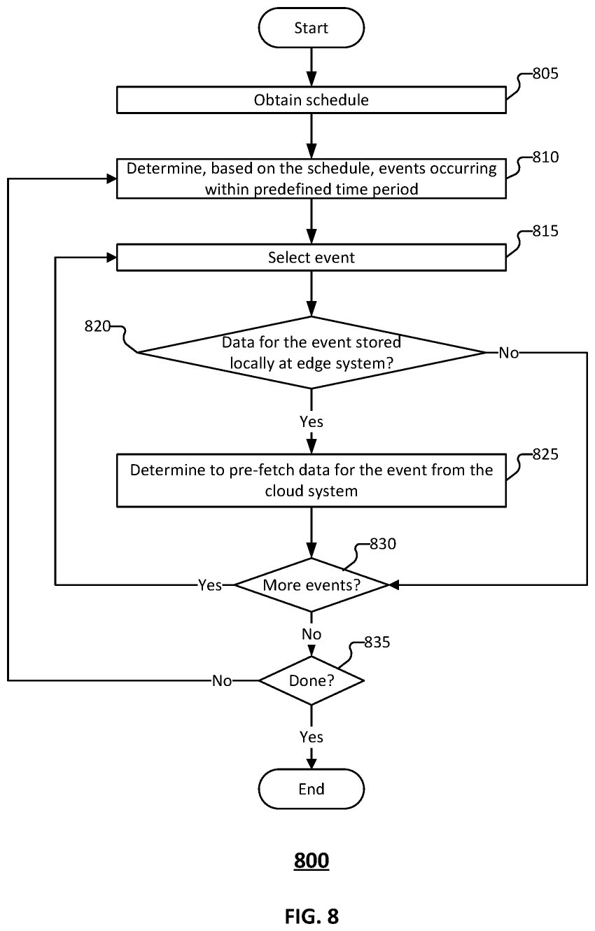

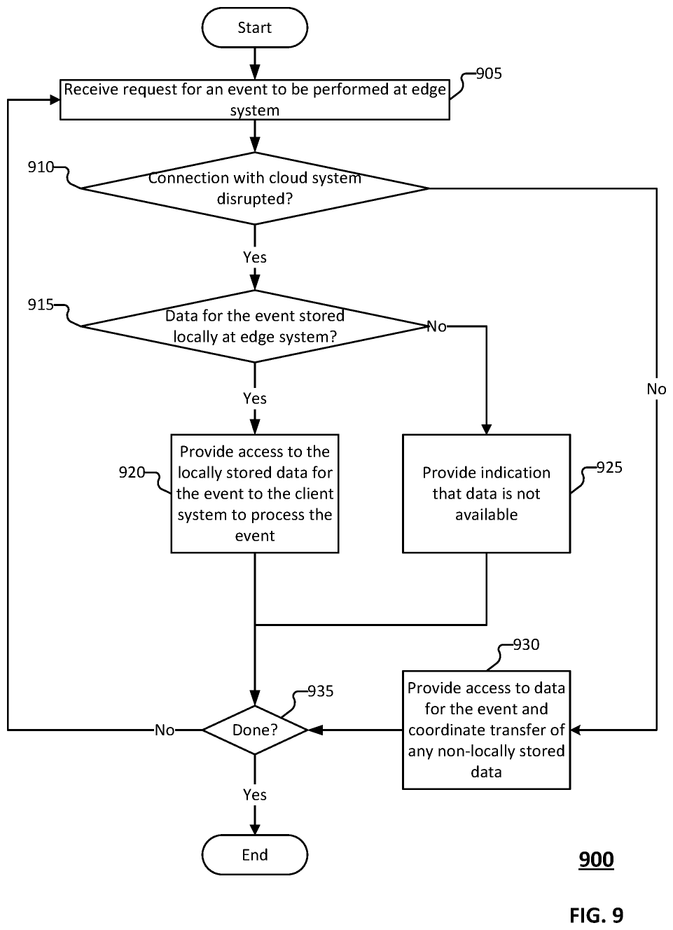

FIG. 1A is a block diagram of a network system according to various embodiments of the present application. According to various embodiments, system 100 is implemented at least in part by system 200 of FIG. 2 and/or system 300 of FIG. 3. In some embodiments, system 100 implements process 400 of FIG. 4, process 500 of FIG. 5, process 600 of FIG. 6, process 700 of FIG. 7, process 800 of FIG. 8, and/or process 900 of FIG. 9.

In some embodiments, a service (e.g., a medical service) is deployed across an edge service and a cloud service. The cloud service may serve as a backend, such as for persisting medical records or routing tasks (e.g., intake workflows, determining a medical provider to analyze a medical record, such as a radiologist to review a certain scan/medical image, etc.). The edge service provides the front-end user experience for a particular task. An example of tasks provided by the edge service is providing medical images to a medical provider for review and receiving input from the medical provider, such as annotations to the patient medical record or medical image, etc. Other examples of tasks provided by the edge service include intake workflows (e.g., scanning a patient quick-response (QR) code and coordinating/scheduling the medical appointments/procedures for the associated user), receiving image data (e.g., medical images) from devices connected to the edge service (e.g., magnetic resonance imaging (MRI) machines, computed tomography (CT) scanning machines, etc.), etc.

In some embodiments, the cloud service performs a smart-routing of tasks. The cloud service determines the users to be assigned certain tasks, such as a radiologist or other medical provider that is tasked with reading the image or medical record, etc. Some physicians may have a preference of certain specialists that are to be assigned tasks for the physician's patients. Accordingly, the cloud service can be configured to learn the physician preferences and automatically assign the tasks according to the physician preferences, etc. The cloud service may train and deploy a machine learning model in connection with one or more of allocating work across users, scheduling tasks or procedures for users such as patients, determining a type of procedure or imaging to be performed based on a medical record or physician notes/annotations, etc. The cloud service can continually train the model to incorporate new preferences or changes in existing preferences, etc.

In the example shown, system 100 comprises cloud service 110 and edge service 120. Cloud service 110 and edge service 120 provide services to one or more client systems (e.g., medical imaging systems, computers used by medical professionals to review lab reports, patient information, medical records, images, studies, etc.). Cloud service 110 and edge service 120 communicate over network 160. In some embodiments, cloud service 110 and edge service 120 provide the service (e.g., application functionality, such as for third party applications) when connected and in the event of connectivity disruption.

In some embodiments, in the event that the connection between cloud service 110 and edge service 120 over network 160 is active, edge service 120 can query data stored on cloud service 110 and obtain data that is not otherwise locally stored at edge service 120. In addition, cloud service 110 and edge service 120 may sync certain data. For example, in response to edge service 120 receiving a new medical image from imaging system 124, edge service 120 syncs this data to cloud service 110 (e.g., to cloud data layer 114) for cloud service 110 to persist such data. Similarly, edge service 120 pre-fetches data stored at cloud service 110 (e.g., data that is not stored locally at edge service 120). Edge service 120 determines data that is expected to be consumed at the edge service side, such as by client system 126 (e.g., to evaluate a medical record, etc.), imaging system 124, etc. Data expected to be consumed is determined based at least in part on one or more pre-fetching criteria, examples of which include data scheduled to be consumed (e.g., according to scheduling information for events to be performed) within a predetermined time threshold, a predetermined amount of time having lapsed since a last sync, an amount of events in the queue to be performed (e.g., the edge service 120 may be configured to maintain the data for M tasks, where M is a positive integer of the overall size of the queue of tasks, or the queue of tasks for a particular user), etc.

In some embodiments, in the event of a connectivity disruption between cloud service 110 and edge service 120, edge service 120 may continue to provide a basic or slimmed down functionality to local devices. For example, edge service 120 may provide to various client systems (e.g., medical imaging systems, computers used by users, etc.) access to data stored locally at edge service 120 despite the connectivity disruption between cloud service 110 and edge service 120. Additionally, edge service 120 may still provide functionality to various applications such as to enable users to perform scheduling functionality, receive images or other studies (e.g., from imaging system 124, etc.), etc. When the connectivity is restored, edge service 120 syncs data generated at the edge service side during the connectivity disruption (e.g., the new medical images, annotations to studies, medical records, images, etc., and new scheduling information).

As illustrated, edge service 120 may include study index 121, edge system 122, imaging system 124, client system 126, and local PACS 128. Local PACS 128 comprises local data store 127 and DICOM comms layer 129.

Edge system 122 provides the services of system 100 to the various other systems that are locally connected to edge service 120, such as imaging system 124, client system 126, and local PACS 128. For example, edge system 122 provides scheduling functionality, order/requisition processing, allocation of orders/requisitions to other services or users (e.g., assign the order to imaging system 124 to obtain an image), etc. Edge system 122 may be implemented by one or more devices, such as an on-premises server(s). Edge system 122 comprises transfer agent 123, which manages/coordinates transfer of data between cloud service 110 and edge service 120. For example, transfer agent 123 determines data to be synced (e.g., new data at edge service 120 to be sent to cloud service 110, or data at cloud service 110 that is to be pre-fetched at edge service 120), negotiates transfer of the data (e.g., with a transfer agent at control layer 116 of cloud service 110), manages a queue of the transfer of data (e.g., a set of records or files), and transfers the data according to associated priority or the queue.

Imaging system 124 is a medical imaging system configured to perform medical imaging services. Examples of medical imaging systems include an MRI scan system, a CT scan system, a radiology system, etc. Edge system 122 can assign/allocate an event, such as to perform a scan, in accordance with a scheduled order/requisition. Imaging system 124 captures the image and transfers the image (or corresponding files) to edge system 122 or local PACS 128. As an example, edge system 122 instantiates a local PACS server to which imaging system 124 sends captured images (e.g., DICOM formatted files). In some embodiments, imaging system 124 is omitted from the edge service—in the event that imaging system 124 is omitted, generation of events to perform a scan takes place in client system 126. In some embodiments, imaging systems are separate from edge service 120 (not shown in FIG. 1) and are in communication with DICOM Comms Layer 129 directly to move images receive image data from the external imaging systems and send image data to the external imaging systems.

In some embodiments, client system 126 is a device used by a user to store, annotate, edit, etc. data stored at edge service 120 (e.g., at local data store 128). Examples of users of client system 126 include: (a) a front-office user to schedule appointments and perform intake of received orders/requisitions; (b) a back-office user to allocate events such as appointments, imaging, study analyses; and (c) a medical professional assigned a particular event such as a patient appointment, an analysis/reading of a medical record (e.g., an image, a lab report, a medical record, etc.), etc.

Local data store 127 is configured to store data locally at edge service 120. For example, local data store 127 stores data generated by imaging system 124, client system 126, and/or edge system 122, or pre-fetched data that is expected to be used. Local data store 127 may store the data in chunks or blocks of chunks, and the data may be deduplicated when stored. Local data store 127 that is a file system with two components: a directed acyclic graph (DAG) storage (e.g., including artifact storage)—where the graph is stored and block storage—where the compressed data are stored with each block representing a collection of chunks. In some embodiments, local data store 127 comprises two separate data stores: a DAG store and a block store. In some embodiments, the local data store 127 stores DICOM files, genetics data, other artifacts, etc. In some embodiments, the block store stores data files in the form of blocks, chunks, files, or any other appropriate format. In some embodiments, local data store 127 stores indices—for example, a DAGindex and a block index.

Study index 121 is used to locate and track data stored in local data store 127. Edge system 122 and/or client system 126 can query study index 121 to determine what is stored in local data store 127. In some embodiments, study index 121 is in communication with indices of local data store 127 (e.g., a DAG index and a block index) to automatically synchronize study index 121 and the local data store 127 stored items.

As illustrated, cloud service 110 comprises cloud system 112 and transfer agent 118.

In some embodiments, cloud system 112 is implemented by one or more servers or virtual machines that instantiate one or more tenants for a plurality of customers/organizations (e.g., medical practices). Cloud system 112 comprises cloud data layer 114, control layer 116, study index 117, and Transfer agent 118. Transfer agent 118 negotiates transfer of data with transfer agent 123 of edge system 122. Control layer 116 controls coordinates tasks and jobs for cloud system 112. For example, control layer 116 makes requests (e.g., send this data or artifact to that edge system, restart this transfer since the transfer has taken longer than a threshold time, etc.). Control layer 116 uses transfer agent 118 and cloud data layer 114 to coordinate transfer of files between different locations (e.g., the cloud storage to an edge storage, an edge storage to cloud storage, etc.). Study index 117 is configured to store information to track and to locate data stored in cloud data layer 114. Cloud data layer 114 is configured to process storage, access, and retrieval queries for cloud stored files and to coordinate storage at other locations including network attached storage (e.g., data store 140). For example, cloud data layer 114 coordinates the partitioning of files into chunks, compressing blocks, files, chunks of data stored at cloud service 110, and/or deduplicating the data stored at cloud service 110. In some embodiments, cloud data layer 114 comprises one or more clusters 115 of virtual machines (e.g., workers) that provide service for a particular tenant. As an example, each tenant for which cloud service 110 provides services has an associated one or more clusters (e.g., different tenants do not share clusters).

System 100 may further include administrator system 130 that is configured to enable a system administrator to configure system 100, such as configuring edge service 120 or cloud service 110.

In some embodiments, system 100 includes fax system 150 or other client system used by a medical professional or patient to submit an order or requisition to cloud service 110 and/or edge service 120.

FIG. 1B is a block diagram illustrating an embodiment of a local data store. In some embodiments, local data store 170 of FIG. 1B implements local data store 127 of FIG. 1A. In the example shown, local data store 170 includes DAG storage 172 (which comprises a DAG index), block storage 176, and block index 178. DAG storage 172 stores DAGs which are used for deduplication. Block storage 176 stores data (e.g., medical data) using blocks, files, chunks, or any other appropriate structure for storing data. Block index 178 stores an index for locating items stored in data storage 176.

FIG. 1C is a block diagram illustrating an embodiment of a cloud data layer. In some embodiments, cloud data layer 180 of FIG. 1C implements cloud data layer 114 of FIG. 1A. In the example shown, cloud data layer 180 includes DAG storage 182 (which comprises a DAG index), block storage 186, and block index 188. DAG storage 182 stores DAGs, which are used for deduplication. Block storage 186 stores data (e.g., medical data) using blocks, files, chunks, or any other appropriate structure for storing data. Block index 188 stores an index for locating items stored in data storage 186. Cloud data layer 180 is implemented using one or more servers or computing units—for example, as part of a cluster with one or more worker machines.

FIG. 2 is a block diagram of an edge system for providing service to client devices according to various embodiments of the present application. According to various embodiments, system 200 implements at least part of system 100 of FIG. 1. In some embodiments, system 200 implements process 400 of FIG. 4, process 500 of FIG. 5, process 600 of FIG. 6, process 700 of FIG. 7, process 800 of FIG. 8, and/or process 900 of FIG. 9.

In some embodiments, the edge service implements system 200 to provide a local service to client systems, imaging systems, or other local devices. System 200 is implemented to provide at least a subset of the service (e.g., the entire service deployed by the edge service and the cloud service) in the event of a disruption in the connection between the edge service and the cloud service. Example services or tasks that may be provided during a connection disruption are scheduling (e.g., scheduling new medical appointments, imaging, etc.), review a patient profile, chart, or medical records (e.g., for tasks that are within a predefined time period or have satisfied another pre-fetching criteria), perform a new (e.g., scheduled) imaging and storing the resulting medical data in association with the patient's record, perform a reading, etc.

If the edge service (e.g., system 200) disconnects from the cloud service, data stored locally at the edge service may return the data to a user upon request (e.g., for consumption such as reviewing and annotating medical records). Additionally, in the face of a disconnection with the cloud service, the edge service may locally store data generated by the edge service or client device (e.g., medical imaging system) connected to the edge service. The newly generated data may be stored at least until the data is synced with the cloud service upon re-connection. For example, the medical device (e.g., medical imaging system) pushes the medical data to system 200, which locally stores the medical data. System 200 partitions the medical data into chunks and transfers the chunks to the cloud service (e.g., subject to deduplication processes that eliminate the redundant transfer of chunks already existing at the cloud service). System 200 may chunk the medical data upon receipt from the medical device (e.g., in the face of a connectivity disruption, system 200 may still generate the chunks in preparation of a syncing upon re-connection). During the connectivity disruption, the edge service may provide the newly generated medical data (e.g., medical data that was generated during the disruption and that has not been synced with the cloud service) to various client systems seeking to digest the medical data.

In the example shown, system 200 implements one or more modules in connection with generating and providing network topology visualizations, etc. System 200 comprises communication interface 205, one or more processors 210, storage 215, and/or memory 220. One or more processors 210 comprises one or more of communication module 225, scheduler module 227, data receiving module 229, data sync determination module 233, pre-fetching module 239, transfer agent 241, and/or user interface module 243.

In some embodiments, system 200 comprises communication module 225. System 200 uses communication module 225 to communicate with various devices, such as imaging systems (e.g., medical imaging systems, such as MRI systems, CT scanning systems, etc.), scheduling systems, client systems (e.g., for a user, such as a medical professional, to use to analyze medical data), and/or administrator systems. For example, communication module 225 provides to communication interface 205 information that is to be communicated (e.g., to another node or client system, or to a corresponding cloud service, etc.). As another example, communication interface 205 provides communication module 225 information received by system 200. Communication module 225 is configured to receive schedule information, event request data (e.g., a medical procedure request), medical data to be stored at the edge service for scheduled procedures or analyses, etc. Communication module 225 is further configured to receive one or more settings or configurations from an administrator. Examples of the one or more settings or configurations include configurations of a machine learning model (e.g., a model used to interpret the natural language query), configuration of system settings such as pre-fetching settings, data compression settings, data chunking settings, edge service-cloud service sync settings, etc.

In some embodiments, system 200 comprises scheduler module 227. System 200 uses scheduler module 227 to perform scheduling for patient visits, medical procedures, medical imaging, and/or physicians (e.g., allocating tasks across physicians, such as assigning a medical image for analysis by a particular medical provider). Scheduler module 227 may be configured to receive an order from a referring physician/medical provider. For example, the referring physician sends to system 200 a fax or other communication. System 200 may be integrated with a fax provider (e.g., via the cloud, etc.). Additionally or alternatively, scheduler module 227 is configured to receive orders/requisitions via email or other electronic means. In response to receiving the order/requisition, scheduler module 227 creates an order to be processed by system 200. Scheduler module 227 can assign a task to one or more users, such as a back-office provider for the medical practice to process the relevant information for the order, such as input or confirm patient information, insurance information, etc. Scheduler module 227 may be configured to automatically perform the scheduling of the procedure, imaging, or review/analysis, or scheduler module 227 may receive the scheduling information from a client system (e.g., a back-office provider) that uses a browser to perform the scheduling.

In some embodiments, system 200 comprises data receiving module 229. In some embodiments, data receiving module 229 differs from communication module 225 in that data receiving module 229 is specifically for receiving data related to medical file data (e.g., image files, video files, medical documents, etc.) or any other file data as opposed to general communication information (e.g., requests, commands, status, etc.). System 200 uses data receiving module 229 to receive data from client systems or other devices that are connected to the edge service or data from a cloud service, such as during a syncing or pre-fetching process in which the edge service is pre-fetching certain medical data for consumption locally (e.g., at a predetermined time or within a predefined time threshold), etc. Examples of data received from client systems or other devices that are connected to the edge service include medical data generated by a medical imaging system or a user such as a medical professional. The medical data generated by the medical imaging system may include image data, such as DICOM files. The medical data generated by the user may include annotations to a patient medical record or other medical data (e.g., a medical image), scheduling information, etc.

In some embodiments, to make the edge service more stable, system 200 maintains a local storage within the browser to store data such as work lists, users, etc. The information stored locally with the browser of the system 200 can be synced with the cloud service when the browser is back online. For example, the client system comprises a set of files (e.g., a set of excel files) that are generated every five mins (or other predefined time period), and the set of files are sent to local storage of the edge device (e.g., the client system) where the set of files are stored on a shared file system (NFS). When system 200 goes offline (e.g., loses connection to the cloud service), system 200 follows a protocol for using data. System 200 opens files in locally available file storage and starts executing off the local storage files (e.g., the set excel files). System 200 uses these locally stored files until connectivity has been restored. When connectivity is restored system 200 syncs its locally stored files with the files stored at the cloud service. As an example, on a day of a scheduled medical exam/appointment, a patient arrives and all the data, studies, modalities, etc. for the medical exam/appointment have been pre-fetched and thus are available from local storage. System 200 schedules a pre-fetching of the applicable medical data to ensure that the medical data is stored locally on the expected date of consumption (e.g., the scheduled day for the exam/appointment). For example, system 200 uses a schedule for each day and pre-fetches the applicable metadata.

In some embodiments, system 200 communicates via communication module 225 and communication interface 205 with a local PACS system. A study index indexes data stored by the Local PACS system for the edge service. The indexed data (e.g., stored in the study index) may include newly generated data (e.g., data ingested from a medical imaging system) or data that has been pre-fetched from the cloud service for local use by a client system (e.g., in anticipation of a certain time or as part of a queue of tasks to be performed by a user, such as a queue of scans to be analyzed by a radiologist). As an example, local PACS system generates a graph representation of all data or chunks stored locally, and the graph (e.g., using a DAG index of the local data store of the local PACS) is stored locally. The study index is an index of what is stored locally at the edge service. When data is to be consumed locally by the edge service or a client system connected to the edge service, system 200 queries the study index to determine whether the data is stored locally (e.g., is stored in a local cache). If the requested data is stored locally, system 200 provides the locally stored data for consumption (e.g., by communicating with the local PACS system to facilitate the client system receiving the appropriate data). Conversely, if the requested data is not stored locally, system 200 causes the receiving of the requested data from the cloud service (e.g., via transfer agent 241) and thereafter provides the requested data for local consumption. In some embodiments, when transfer agent 241 negotiates transfer of the requested data (e.g., with a transfer agent disposed on the cloud service), transfer agent 241 and the cloud service perform a fine grain negotiation (e.g., based at least in part on the edge index and/or the cloud index) to determine what specifically needs to be transferred (e.g., what subset of chunks for the medical data) to enable the edge service to store the requested data.

In some embodiments, the edge service stores the data (e.g., medical data) in chunks and/or blocks. In connection with storing the data in chunks the edge service indexes (e.g., as a study index) the specific chunks associated with a particular medical record file (e.g., a medical record, an image, or other study). For example, the study index handles the chunkification of the data to identify the relevant chunks to be used to consume certain medical data. In some embodiments, the study index does not know the specifics of the Storage (e.g., DAG, chunks, blocks, etc.), and the study index only keeps a mapping between the patient information (e.g., a medical record) and snapshot identifiers (e.g., gW9uWDFY9HZPgiYh9QcyWH4kQvNjMALGgaqHd9xpQRBBv5). For example, the study index can execute queries like: give me all snapshot identifiers for MRI scans of patient A. Additionally, edge service indexes (e.g., as a logical index) information pertaining to the medical data (e.g., the image file) and various other metadata that can be queried. For example, the medical image in DICOM file format comprises raw imaging data and metadata, such as metadata pertaining to the order for which the medical image was generated, the physician, the modality for generating the medical image, demographics, patient information, etc. In response to receiving the medical data in DICOM file format, the edge service digests the file and generates/updates a logical index for the information (e.g., the metadata) for the medical data. The logical index may be used to associate studies, raw data, and various metadata that can be queried.

In some embodiments, system 200 comprises data sync determination module 233. System 200 uses data sync determination module 233 to determine when/whether to sync the locally stored data and the data persisted at the cloud service. Data sync determination module 233 determines to sync the locally stored data and the data stored at the cloud service based at least in part on one or more of: (i) the occurrence of a predefined event, (ii) the expiration of a predetermined time period from the time since the last sync, (iii) a predetermined schedule, (iv) an amount of new data generated at either the edge service or the cloud service exceeding a threshold amount of data (e.g., number of records, images, etc.) since a last sync, etc. Examples of a predefined event which may trigger a sync include reconnection between the edge service and the cloud service, receipt of a user request to perform a sync, receipt of new medical data at the edge service such as a new medical image from a client system connected to the edge service, etc.

In some embodiments, edge service partitions data into chunks of data (e.g., to partition each file into constituent chunks). As an example, the chunks may have a size of 256 kB. However, various other chunk sizes may be implemented. The edge service can be configured to chunk a file upon receipt of the file (e.g., from a client system), according to a predetermined schedule/frequency, or when the edge service and cloud service are to be synced (or at least when the edge service and cloud service are to negotiate the specific chunks to be transferred to perform the sync). The edge service stores a unique identifier associated with each chunk (e.g., in a study index or in the DAG). As an example, the unique identifier is obtained by performing a hash against a particular chunk. Accordingly, identical chunks can be identified based on the unique identifier or hash, which can be used to determine whether a particular chunk is stored at both the edge service and the cloud service. In some embodiments, the hashes of each chunk are stored in the DAG not in the study index.

In some embodiments, the edge service generates a Merkle Directed Acyclic Graph (DAG) for the files or chunks stored at the edge service. The edge service uses snapshots—for example, a Merkle DAG graph—for the chunks to identify new chunks or chunks that have been deleted or modified. For example, the edge service uses snapshots to capture a state of the data at a particular time and the tree/graph is updated to include information from earlier snapshots, such as to include or deduplicate data from a second snapshot. In some implementations, the edge service is configured so that subsequent snapshots created from an earlier snapshot (e.g., a snapshot of the same directory) have all the information from the earlier snapshot. The edge service generates or maintains an index of snapshots and information comprised in each snapshot. In some embodiments, the edge service as part of the local data store of the local PACS will 1) using a data chunking module create chunks, 2) create the DAG using the chunks, 3) save the DAG in a DAG store (e.g., of an artifact storage of the local data store of the local PACS), 4) create and compress blocks from the chunks, and 5) save each block in a block store (e.g., of a data storage of the local data store of the local PACS).

The size of data into which an edge service data chunking module partitions the data can be configurable. If chunks are very small, edge service can identify all types of duplicates (e.g., the extent of the deduplication possible is very high), however, if the chunks are too small, the administration for managing chunks becomes large. In some embodiments, edge service uses compressions to be able to leverage larger chunks to reduce the administration costs for managing/indexing chunks. For example, the files are partitioned into chunks having a size of 256 kB, and then edge service compresses each chunk. The computing of the Merkle DAG graph before compression of the chunks decouples the deduplication from the compression, thereby ensuring that the graph is always the same regardless of the compression algorithm being implemented. Various types of chunking mechanisms can be implemented, such as content-defined chunking or fixed-size chunking. In some embodiments, for DICOM formatted files fixed-size chunking is used as there is little benefit for content-defined chunking because the files are very small.

Chunkification of the data stored at the edge service (e.g., the medical data) enables the edge service and the cloud service to use the chunks for a file-to-be-synced as a basis for negotiating what specific chunks or blocks (e.g., sets of chunks) are to be transferred. Chunkification allows for a very fine determination of a delta between files (e.g., a specific set of identified chunks) to be uploaded (or downloaded), which leads to efficiency gains. Further, the fine granularity of chunkification enables edge service to implement deduplication for local storage of data.

In some embodiments, the edge service uses a data compression module to compress the data stored at the edge service, such as chunks generated by a data chunking module. The data compression module may be configured to implement a standard compression technique (e.g., such as creating a .zip file for the particular chunk/data) or using video encoders to compress the data/chunks, which takes advantage that two images next to each other are very alike.

In some embodiments, the edge service implements the generation of blocks respectively comprising one or more chunks. Because chunks are very small, blocks can be used to reduce the administration for managing a large number of small files. The edge service organizes the chunks into a set of blocks (e.g., the system bunches a plurality of chunks to make a block), and then the chunks can be compressed for storage. If the edge service implements the storage of blocks comprising a set of chunks, the edge service uses an indexing module to maintain/generate an index that controls/indexes data that is to be compressed, such as maintaining an index or mapping of chunks to blocks. Each block is associated with the applicable snapshot, and each block is compressed and stored in a different data storage. The size of blocks can be configurable based on user/system preferences. As an example, the edge service is configured to use blocks having a size of 64 MB. In some implementations, the size of blocks can be dynamic such that a particular block can have a different size than another block.

In some embodiments, in response to a determination to save a file to storage (e.g., to the cloud storage), the edge service first computes the graph, sends the graph to storage, and the storage uses the graph to determine which chunks are already stored on the storage. For example, edge service determines which blocks/chunks are already in the graph and only transfer those that are not already stored (e.g., stored on the cloud).

In some embodiments, system 200 comprises pre-fetching module 239. System 200 uses pre-fetching module 239 to pre-fetch files (e.g., medical data) stored at the cloud service for use locally at the edge service (e.g., by a client system connected to the edge service). Pre-fetching module 239 can coordinate the pre-fetching of files based on an anticipated use of the files at the edge service or according to a predefined schedule. As an example, if a particular radiologist is assigned a queue of scans/medical images to be performed over the next month, pre-fetching module 239 can determine the set of scans/medical images to be stored locally for access by the radiologist over that time. The predetermined time or number of tasks (e.g., scan reviews) used by pre-fetching module 239 to determine the files (e.g., scans/medical images) to be stored locally at the edge service (e.g., confirmed to be currently stored or, if not currently stored, retrieved from the cloud service) may be configurable. For example, pre-fetching module 239 determines to pre-fetch files expected to be used/needed in the next 48 hours. Various other time periods may be implemented (e.g., 24 hours, a week, a month, etc.). As another example, pre-fetching module 239 determines to pre-fetch the next N files (e.g., N being a positive integer) to be used by a user or set of users. In response to determining that a file is to be pre-fetched, pre-fetching module 239 provides an indication to transfer agent 241 that the file is to be retrieved from cloud service (e.g., the file is added to the queue of files to be synced/downloaded).

An example scenario includes the workflow for a particular patient for which the system has received and scheduled an order for study (e.g., a medical imaging). A medical imaging system produces DICOM files corresponding to the study itself and the DICOM file for the study is uploaded to the cloud service. Once the study is uploaded to the cloud service and available for reading, reading/analysis of the study is assigned to a user/device for reading. The assignment of a particular study is performed as early as possible or at least a threshold amount of time before the study is expected to be read so the edge service (e.g., the device being used by the physician to which the study is assigned) can do pre-fetching early enough to ensure that the user can still process a queue of tasks (e.g., a queue of studies to be read). In some embodiments, in addition to pre-fetching the image or lab report for the study, the edge service prefetches other medical data for the patient, such as the patient's chart/medical records, other or previous studies performed for the patient, etc.

In some embodiments, system 200 comprises transfer agent 241. System 200 uses transfer agent 241 to negotiate transfer of data with a companion transfer agent on another device, such as a cloud service or a client system that is sending files (e.g., medical images) to system 200 or accessing files stored at system 200. Transfer agent 241 receives an indication (e.g., from another module of system 200) of a file/record to be stored at the cloud service or retrieved from the cloud service, and transfer agent 241 communicates with the companion transfer agent on the cloud service to determine the specific chunks or blocks that are to be sent to, or received from, the cloud service for the file(s) requested. For example, the edge service (e.g., transfer agent 241) and the cloud service determine the delta between the data (at least with respect to the requested files) that is stored at the edge service and the data stored at the cloud service, such as based on the edge index maintained by indexing module 231 and the cloud index stored at the cloud service. In some embodiments, transfer agent 241 only transfers those chunks/blocks corresponding to the determined delta between the data stored at the edge service and the data stored at the cloud service. Transfer agent 241 may implement an http-based transfer protocol that guarantees delivery of the applicable data between the edge service and the cloud service (e.g., the transfer agent 241 is configured to guarantee that transfers are efficient, reliable, and safe).

In connection with syncing a particular file across the edge service and the cloud service, system 200 obtains the input data, deduplicates the data, compresses the data, negotiates with the cloud service (e.g., the transfer agent for the cloud service), and then transfers piece by piece (e.g., block by block or chunk by chunk).

In some embodiments, the edge service does not fully replicate what is stored at the cloud service. For example, the edge index does not fully replicate the cloud index. Rather, the edge service stores locally a small fraction of what is stored at the cloud service. In some embodiments, data generated locally at the edge service is synced with the cloud service. The syncing is often delayed, so in some instances the edge service stores data that is not already in the cloud service. The newly generated data is synced with the cloud service according to a predetermined interval (e.g., at the end of the business day) or in response to a particular event occurring. Another example is of data generated at the edge service requiring (eventual) sync with the cloud service annotation or modifications to scans, medical records, etc. by a medical provider (e.g., a medical provider performing a task assigned for the particular patient/study). Generally, the syncing of data stored on the cloud service that is not already stored on the edge service is performed when/in connection with the data being needed at the edge service for local consumption (e.g., for a client system to access/modify the data).

In some embodiments, client systems connected to the edge device also have transfer agents. For example, medical imaging systems have a light-weight deployment of an image transfer layer (ITL) agent that negotiates with transfer agent 241 to store the data at the edge service (e.g., at system 200). Transfer agent 241 receives images from the medical imaging systems, stores the images in local storage, organizes transfer of images to the cloud service, and then pushes the images to the cloud when connected.

In some embodiments, transfer agent 241 is governed by state machines that are aware of connectivity status. Accordingly, transfer agent 241 is used to determine whether the edge service is connected to the cloud service. In response to determining that the connection between the edge service and the cloud service is disrupted, transfer agent 241 queues files that are determined to be synced with the cloud service, and transfer agent 241 works to initiate the transfers upon the connection being restored.

Transfer agent 241 may transfer data to be synced based on a priority. As an example, data stored locally at the edge service to be synced to the cloud service may have a different priority than data to be retrieved from the cloud service (e.g., the data stored locally to be synced may have a higher priority because it is more important to ensure that the requisite data is stored at the edge service for assignment of tasks/processing). As another example, the priority may be based at least in part on the expected date/time that the data is to be consumed locally (e.g., at the edge service or client system connected to the edge service).

In some embodiments, system 200 comprises user interface module 243. System 200 uses user interface module 243 to configure and provide a user interface to a user, such as to a client system used by a user (e.g., a medical professional using the edge system to access medical records for a procedure or analysis). User interface module 243 configures a user interface to provide medical data in response to a request from a user, such as a patient profile, imaging data, etc. As an example, system 200 configures a user interface via which a user (e.g., a medical professional) can review and/or annotate the medical data, etc.

According to various embodiments, storage 215 comprises one or more of file system data 260, medical data 265, and index 270. Storage 215 comprises a shared storage (e.g., a network storage system) and/or database data, and/or user activity data. In some embodiments, file system data 260, medical data 265, and/or index 270 may be stored as encrypted data. The encryption for file system data 260, medical data 265, and/or index 270 may be performed at the application level.

In some embodiments, file system data 260 comprises a database such as one or more datasets (e.g., one or more datasets for patients, imaging data, schedule data, etc.).

Medical data 265 comprises information pertaining to one or more patients or potential patients. Examples of information comprised in medical data 265 include procedure/image requisition, a patient profile, protected health information (PHI), image data (e.g., imaging performed by a medical imaging system connected to system 200), medical evaluation data (e.g., results from an evaluation performed by a user of a client system connected to system 200), etc. Various other types of medical data may be implemented.

Index 270 comprises information pertaining to one or more records (e.g., medical records, requisitions, results, etc.). Index 270 comprises an index (e.g., an edge index) of data stored at the edge system (e.g., at system 200). For example, index 270 comprises an index for data that can be accessed locally at the edge service (e.g., by querying system 200) in the event of a disruption in the connection between the edge service and the cloud service, or newly acquired data during such disruption (e.g., image data or schedule data that is input/received during the disruption). Examples of index 270 include an index of records stored at system 200, an index of chunks stored at system 200 for various records, etc.

According to various embodiments, memory 220 comprises executing application data 275. Executing application data 275 comprises data obtained or used in connection with executing an application such as an application to determine or predict when to pre-fetch data from a cloud service to make available at the edge system in the event of connection disruption, an application to queue data (e.g., chunks of data) that is to be transferred to the cloud service, etc. Other applications comprise any other appropriate applications (e.g., an index maintenance application, a communications application, a report preparation application, a user interface application, a data analysis application, an anomaly detection application, a user authentication application, a security policy management/update application, a medical imaging application, etc.).

FIG. 3 is a block diagram of a cloud system for providing service to client devices via an edge service according to various embodiments of the present application. According to various embodiments, system 300 implements at least part of system 100 of FIG. 1. In some embodiments, system 300 implements process 400 of FIG. 4, process 500 of FIG. 5, process 600 of FIG. 6, process 700 of FIG. 7, process 800 of FIG. 8, and/or process 900 of FIG. 9.

In the example shown, system 300 implements one or more modules in connection with generating and providing network topology visualizations, etc. System 300 comprises communication interface 305, one or more processors 310, storage 315, and/or memory 320. One or more processors 310 comprises one or more of communication module 325, scheduler module 327, data receiving module 329, indexing module 331, data sync determination module 333, data chunking module 335, data compression module 337, transfer agent 339, data deduplicating module 341, and/or user interface module 343.

In some embodiments, the cloud service implements system 300 to provide full/core functionality for a service, such as a software as a service, to one or more tenants (e.g., customers such as medical practices) that comprise one or more edge services and a plurality of client systems that connect to the one or more edge services to use the software as a service.

In some embodiments, system 300 comprises communication module 325. System 300 uses communication module 325 to communicate with various devices, such as one or more edge services (e.g., tenants for different medical practices, etc.), scheduling systems, client systems (e.g., for a user, such as a medical professional, to use to analyze medical data), and/or administrator systems. For example, communication module 325 provides to communication interface 305 information that is to be communicated (e.g., to another node or client system, or to a corresponding cloud service, etc.). As another example, communication interface 305 provides to communication module 325 information received by system 300. Communication module 325 is configured to receive a schedule information, event request data (e.g., a medical procedure request), medical data to be stored at the edge service for scheduled procedures or analyses, etc. Communication module 325 is further configured to receive one or more settings or configurations from an administrator. Examples of the one or more settings or configurations include configurations of a machine learning model (e.g., a model used to interpret the natural language query), configuration of system settings such as pre-fetching settings, data compression settings, data chunking settings, edge service-cloud service sync settings, etc.

In some embodiments, system 300 comprises scheduler module 327. System 300 uses scheduler module 327 to perform scheduling for patient visits, medical procedures, medical imaging, and/or physicians (e.g., allocating tasks across physicians, such as assigning a medical image for analysis by a particular medical provider). Scheduler module 327 may be configured to receive data, such as an order for a patient appointment or other task, from the edge service which had received the order from a referring physician/medical provider. In response to receiving the data, scheduler module 327 creates an order to be processed by system 300. Scheduler module 327 can assign a task to one or more users, such as a back-office provider for the medical practice to process the relevant information for the order, such as input or confirm patient information, insurance information, etc. Scheduler module 327 may be configured to automatically perform the scheduling of the procedure, imaging, or review/analysis, or scheduler module 327 may receive the scheduling information from a client system (e.g., a back-office provider) that uses a browser to perform the scheduling. In some embodiments, in response to receiving from the edge service a medical image captured from a medical imaging system, scheduler module 327 may associate the medical image with a particular patient's medical record and allocate a task to a medical provider or other service. For example, scheduler module 327 allocates a study (e.g., scan analysis) to a radiologist, such as based on workload, qualifications, or user or organization preferences (e.g., preferences of the referring doctor, or preferences of the doctor to whom the task is to be assigned), etc.

In some embodiments, system 300 comprises data receiving module 329. System 300 uses data receiving module 329 to receive data from an edge service(s) or client systems or other devices that are connected to the edge service, such as during a syncing or pre-fetching process in which the edge service is pre-fetching certain medical data for consumption locally (e.g., at a predetermined time or within a predefined time threshold), etc. Examples of data received from the edge service or client systems or other devices that are connected to the edge service include medical data generated by a medical imaging system or a user such as a medical professional. The medical data generated by the medical imaging system may include image data, such as DICOM files. The medical data generated by the user may include annotations to a patient medical record or other medical data (e.g., a medical image), scheduling information, etc.

In some embodiments, system 300 comprises indexing module 331. System 300 uses indexing module 331 to index the data stored at the cloud service. The indexed data (e.g., a cloud index) may include newly generated data (e.g., data ingested from a medical imaging system) or data that has been synced with one or more edge services. In some embodiments, the cloud service provides service to a plurality of tenants (e.g., different customers, medical practices, etc.), and each tenant has a corresponding cloud index that indexes data for that tenant. As an example, indexing module 331 generates a graph representation of all data or chunks stored at the cloud service, and the graph (e.g., the index) is stored at the cloud service. When data is to be consumed locally by the edge service or a client system connected to the edge service (e.g., for data not locally stored at the edge service), the edge service or client system queries the cloud service for the data, and the cloud service in turn queries the cloud index to determine whether the data is stored at the cloud service or whether a newer/updated version of the data is stored at the cloud service which is to be synced with the edge service for local consumption.

In some embodiments, system 300 comprises data sync determination module 333. System 300 uses data sync determination module 333 to determine when/whether to sync the data persisted at the cloud service with an edge service or another client system. Data sync determination module 333 determines to sync the data persisted at the cloud service with an edge service or another client system based at least in part on one or more of (i) the occurrence of a predefined event, (ii) the expiration of a predetermined time period from the time since the last sync, (iii) a predetermined schedule, (iv) an amount of new data generated at either the edge service or the cloud service exceeding a threshold amount of data (e.g., number of records, images, etc.) since a last sync, etc. Examples of a predefined event which may trigger a sync include reconnection between the edge service and the cloud service, receipt of a user request to perform a sync, receipt of new medical data at the edge service such as a new medical image from a client system connected to the edge service, etc.

In some embodiments, system 300 comprises data chunking module 335. System 300 uses data chunking module 335 to partition data into chunks of data (e.g., to partition each file into constituent chunks). As an example, the chunks may have a size of 256 kB. However, various other chunk sizes may be implemented. Data chunking module 335 may identify chunks in data received from an edge service, such as by deconstructing blocks transferred/synced from the edge service. In some embodiments, data chunking module 335 can be configured to chunk a file upon receipt of the file (e.g., from an edge service), according to a predetermined schedule/frequency, or when the edge service and cloud service are to be synced (or at least when the edge service and cloud service are to negotiate the specific chunks to be transferred to perform the sync). System 300 stores a unique identifier associated with each chunk. As an example, the unique identifier is obtained by performing a hash against a particular chunk. Accordingly, identical chunks can be identified based on the unique identifier or hash, which can be used to determine whether a particular chunk is stored at both the edge service and the cloud service.

In some embodiments, data chunking module 335 chunks data according to the same technique or configurations as the edge service (e.g., data chunking module 235 of system 200).

In some embodiments, system 300 comprises data compression module 337. System 300 uses data compression module 337 to compress the data stored at the cloud service, such as chunks generated by data chunking module 335. Data compression module 337 may be configured to implement a standard compression technique, such as creating a .zip file for the particular chunk/data, or using video encoders to compress the data/chunks, which takes advantage that two images next to each other are very alike.

In some embodiments, system 300 comprises transfer agent 339. System 300 uses transfer agent 339 to negotiate transfer of data with a companion transfer agent on another device, such an edge service or a client system that is sending files (e.g., medical images) to the edge service or accessing files stored at the edge service. In some embodiments, transfer agent 339 is the same as, or configured similar to, the transfer agent(s) of the edge service (e.g., transfer agent 241 of system 200).

Transfer agent 339 receives an indication (e.g., from another module of system 300) of a file/record to be synced with the edge service, and transfer agent 339 communicates with the companion transfer agent on the cloud service to determine the specific chunks or blocks that are to be sent to, or received from, the edge service for the file(s) to be synced or provided for local consumption at the edge service. For example, the cloud service (e.g., transfer agent 339) and the edge service determine the delta between the data (at least with respect to the requested files) that is stored at the edge service and the data stored at the cloud service, such as based on the cloud index maintained by indexing module 331 and the edge index stored at the edge service. In some embodiments, transfer agent 339 only transfers those chunks/blocks corresponding to the determined delta between the data stored at the edge service and the data stored at the cloud service. Transfer agent 339 may implement an http-based transfer protocol that guarantees delivery of the applicable data between the edge service and the cloud service (e.g., the transfer agent 339 is configured to guarantee that transfers are efficient, reliable, and safe).

In some embodiments, system 300 comprises data deduplicating module 341. System 300 uses data deduplicating module 341 to deduplicate data stored at the cloud service. For example, data deduplicating module 341 identifies files or parts of the files (e.g., chunks, blocks, etc.) that are duplicate to files or parts of files already stored at the cloud service, and data deduplicating module 341 stores a single version of the file or part of the file. Data deduplicating module 341 may use indexing module 331 to update an index of chunks to file(s) based at least in part on the deduplication of certain chunks, etc.

In some embodiments, system 300 comprises user interface module 343. System 300 uses user interface module 343 to configure and provide (e.g., via the edge service) a user interface to a user, such as to a client system used by a user (e.g., a medical professional using the edge system to access medical records for a procedure or analysis). User interface module 343 configures a user interface to provide medical data in response to a request from a user, such as a patient profile, imaging data, etc. As an example, system 300 configures a user interface via which a user (e.g., a system administrator) can configure the service, such as to configure security policies, access policies, synchronization policies, etc.

According to various embodiments, storage 315 comprises one or more of file system data 360, medical data 365, and index 370. Storage 315 comprises a shared storage (e.g., a network storage system) and/or database data, and/or user activity data. In some embodiments, file system data 360, medical data 365, and index 370 may be stored as encrypted data. The encryption for file system data 360, medical data 365, and index 370 may be encrypted at rest (e.g., the encryption is performed at the storage level).

In some embodiments, file system data 360 comprises a database such as one or more datasets (e.g., one or more datasets for patients, imaging data, schedule data, etc.).

Medical data 365 comprises information pertaining to one or more patients or potential patients. Examples of information comprised in medical data 365 includes procedure/image requisition, a patient profile, protected health information (PHI), image data (e.g., imaging performed by a medical imaging system connected to system 300), medical evaluation data (e.g., results from an evaluation performed by a user of a client system connected to system 300), etc. Various other types of medical data may be implemented. In some embodiments, system 300 is used to persist the medical data (e.g., as medical data 365) because medical data stored locally at the edge service may be purged after a predetermined amount of time after a corresponding event (e.g., a medical imaging procedure, a medical evaluation/analysis by a medical professional) or after the medical data generated (e.g., newly received by) at the edge service is synced with medical data stored at system 300.

Index 370 comprises information pertaining to one or more records (e.g., medical records, requisitions, results, etc.). Index 370 comprises an index (e.g., an edge index) of data stored at the cloud system (e.g., at system 300). For example, index 370 comprises an index for data that is synced with an edge service (e.g., by querying system 300) for the use locally at the edge service in the event of a disruption in the connection between the edge service and the cloud service. Examples of index 370 include an index of records stored at system 300, an index of chunks stored at system 300 for various records, etc.

According to various embodiments, memory 320 comprises executing application data 375. Executing application data 375 comprises data obtained or used in connection with executing an application such as an application to determine or predict when to pre-fetch data from a cloud service to make available at the edge system in the event of connection disruption, an application to queue data (e.g., chunks of data) that is to be transferred to the edge service, etc. Other applications comprise any other appropriate applications (e.g., an index maintenance application, a communications application, a report preparation application, a user interface application, a data analysis application, an anomaly detection application, a user authentication application, a security policy management/update application, a medical imaging application, etc.).

FIG. 4 is a flow diagram of a method for providing access to data at an edge system according to various embodiments of the present application. In some embodiments, process 400 is implemented by system 100 of FIG. 1. Process 400 may be implemented by system 200 of FIG. 2 and/or edge service 120 of system 100.

At 405, the system connects to a cloud system. At 410, the system maintains an edge index of data stored locally at the edge system (e.g., the edge data). The edge data may be newly generated, such as based on scheduling or imaging activities performed at the edge service (e.g., from a client system, medical imaging system, etc. connected to the edge service). Additionally or alternatively, the edge data may, at 415, sync at least a subset of data comprised at the edge system and the cloud system. In some embodiments, the at least the subset of data is synced based at least in part on the edge index. At 420, the system provides access to the data stored locally at the edge system to one or more client systems connected to the edge system. At 425, a determination is made as to whether process 400 is complete. In some embodiments, process 400 is determined to be complete in response to a determination that no further events (e.g., medical imaging or medical data analysis) are to be processed, a connection between the edge system and cloud system is disrupted, a user has exited the system, an administrator indicates that process 400 is to be paused or stopped, etc. In response to a determination that process 400 is complete, process 400 ends. In response to a determination that process 400 is not complete, process 400 returns to 405.

FIG. 5 is a flow diagram of a method for managing data at a cloud system for use by an edge service according to various embodiments of the present application. In some embodiments, process 500 is implemented by system 100 of FIG. 1. Process 500 may be implemented by system 300 of FIG. 3 and/or cloud service 110 of system 100.

At 505, the system connects to an edge system. At 510, the system maintains a cloud index of data stored locally at the cloud system. At 515, the system syncs at least a subset of data comprised at the edge system and the cloud system. In some embodiments, the at least the subset of data is synced based at least in part on the cloud index. At 520, a determination is made as to whether process 500 is complete. In some embodiments, process 500 is determined to be complete in response to a determination that no further events (e.g., medical imaging or medical data analysis) are to be processed, a connection between the edge system and cloud system is disrupted, a user has exited the system, an administrator indicates that process 500 is to be paused or stopped, etc. In response to a determination that process 500 is complete, process 500 ends. In response to a determination that process 500 is not complete, process 500 returns to 505.

FIG. 6 is a flow diagram of a method for transferring data between an edge system and a cloud system according to various embodiments of the present application. In some embodiments, process 600 is implemented by system 100 of FIG. 1. Process 600 may be implemented by system 200 of FIG. 2 and/or system 300 of FIG. 3. Process 600 may be invoked by process 400 of FIG. 4, such as at 415.

At 605, the system determines to transfer data for a particular record, for example, a medical record.

At 610, the system determines data for the particular record stored at the edge system. At 615, the system determines data for the particular record stored at the cloud system. At 620, the system determines whether the data stored at the edge system and the data stored at the cloud system for the particular record are different. For example, the system determines whether data at one of the edge system or cloud system has been changed since a last sync, or whether the entire data for a record is stored at both the edge system and the cloud system. In response to determining that the data stored at the edge system and the data stored at the cloud system for the particular record are different, process 600 proceeds to 625. Conversely, in response to determining that the data stored at the edge system and the data stored at the cloud system for the particular record are not different, process 600 proceeds to 630. At 625, the system updates the transfer queue to include the difference between the data stored at the edge system and the data stored at the cloud system for the particular record. The transfer agents for the edge system and cloud system coordinate the transfer of data in the transfer queue. At 630, a determination is made as to whether process 600 is complete. In some embodiments, process 600 is determined to be complete in response to a determination that no further events (e.g., medical imaging or medical data analysis) are to be processed, an event for a particular record is complete, a connection between the edge system and cloud system is disrupted, a user has exited the system, an administrator indicates that process 600 is to be paused or stopped, etc. In response to a determination that process 600 is complete, process 600 ends. In response to a determination that process 600 is not complete, process 600 returns to 605.

FIG. 7 is a flow diagram of a method for storing data according to various embodiments. In some embodiments, process 700 is implemented by system 100 of FIG. 1. Process 700 may be implemented by system 200 of FIG. 2 and/or system 300 of FIG. 3. Process 700 may be implemented in connection with storing data, such as storing newly generated/received data at the edge service.

At 702, the system receives data for a particular record. For example, the edge service receives a file(s) generated based on a medical imaging. At 704, the system partitions the data for the particular record into a plurality of chunks. At 706, a hash is computed for each of the chunks. In 708, a DAG is created using the chunks. In 710, the DAG is saved in a DAG store. At 712, the chunks not already stored (a deduplication step) are grouped in blocks. At 714, the blocks are compressed. At 716, the blocks are stored in a block store. At 720, a determination is made as to whether process 700 is complete. In some embodiments, process 700 is determined to be complete in response to a determination that no further events (e.g., medical imaging or medical data analysis) are to be processed, no further data is received, an event for a particular record is complete, a user has exited the system, an administrator indicates that process 700 is to be paused or stopped, etc. In response to a determination that process 700 is complete, process 700 ends. In response to a determination that process 700 is not complete, process 700 returns to 702.

FIG. 8 is a flow diagram of a method for prefetching data to be stored at the edge system in advance of an event according to various embodiments. In some embodiments, process 800 is implemented by system 100 of FIG. 1. Process 800 may be implemented by system 200 of FIG. 2 and/or edge service 120 of system 100. Process 800 may be implemented in connection with pre-fetching certain data from the cloud service, such as to enable functionality with respect to such data (e.g., medical imaging, reading/annotating studies, etc.) even in the case of connectivity disruptions with the cloud service.

At 805, the system obtains a schedule. For example, the system obtains schedule information pertaining to the scheduling of one or more events such as medical appointments, patient imaging, medical record reading/analyzing, etc. At 810, the system determines, based at least in part on the schedule, events occurring within a predefined time period. At 815, the system selects an event (e.g., from the events identified to be occurring within the predefined time period). At 820, the system determines whether data for the event is stored locally at the edge system/edge service. For example, the system determines the requisite data for the event (e.g., a medical record or patient information, a study, a lab report) and then determines whether the data is stored locally. In response to determining that the data for the event is stored locally, process 800 proceeds to 825. Conversely, in response to determining that the data for the event is not stored locally, process 800 proceeds to 830. At 825, the system determines to pre-fetch data for the event from the cloud system. The system may update a transfer queue (e.g., managed by a transfer agent). In some embodiments, 825 invokes a service or process to pre-fetch the data. At 830, the system determines whether a set of events occurring within the predefined time period are to be analyzed. In response to determining that another event is to be analyzed, process 800 returns to 815 and process 800 iterates over 815-830 until no further events are to be evaluated. At 835, a determination is made as to whether process 800 is complete. In some embodiments, process 800 is determined to be complete in response to a determination that no further events (e.g., medical imaging or medical data analysis) are to be processed, no further data is received, an event for a particular record is complete, a user has exited the system, an administrator indicates that process 800 is to be paused or stopped, etc. In response to a determination that process 800 is complete, process 800 ends. In response to a determination that process 800 is not complete, process 800 returns to 810.

FIG. 9 is a flow diagram of a method for providing service at an edge system based on a connection between the edge system and the cloud system according to various embodiments. In some embodiments, process 900 is implemented by system 100 of FIG. 1. Process 900 may be implemented by system 200 of FIG. 2 and/or edge service 120 of system 100.

At 905, the system receives a request for an event to be performed at the edge system. For example, the system receives a request to perform a medical imaging using a medical imaging system connected to the edge system. As another example, the system receives a request from a radiologist to review a study or to annotate the study, etc.

At 910, the system determines whether the connection with the cloud system is disrupted. For example, the transfer agent comprised in the edge system detects whether the edge system is connected to the cloud service. In response to determining that the connection with the cloud system is disrupted, process 900 proceeds to 915. Conversely, in response to determining that the connection with the cloud system is not disrupted, process 900 proceeds to 930.

At 915, the system determines whether data for the event is stored locally at the edge system. For example, the system determines the requisite data for the event and determines whether all such data is stored locally. In response to determining that the data for the event is stored locally at the edge system, process 900 proceeds to 920. Conversely, in response to determining that the data for the event is not stored locally at the edge system, process 900 proceeds to 925.

At 920, the system provides access to the locally stored data for the event to the client system to process the event.

At 925, the system provides an indication that the data is not available. For example, if a radiologist requested a particular study, the system indicates that such study is unavailable as a result of the connectivity disruption.

At 930, the system provides access to data for the event and coordinates transfer of any non-locally stored data. For example, the system determines the requisite data for the event, determines whether any such data is stored locally, and whether any additional information is to be retrieved from the cloud system.

At 935, a determination is made as to whether process 900 is complete. In some embodiments, process 900 is determined to be complete in response to a determination that no further events (e.g., medical imaging or medical data analysis) are to be processed, no further data is received, an event for a particular record is complete, a user has exited the system, an administrator indicates that process 900 is to be paused or stopped, etc. In response to a determination that process 900 is complete, process 900 ends. In response to a determination that process 900 is not complete, process 900 returns to 905.

Various examples of embodiments described herein are described in connection with flow diagrams. Although the examples may include certain steps performed in a particular order, according to various embodiments, various steps may be performed in various orders and/or various steps may be combined into a single step or in parallel.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system for providing a service to a set of client systems, comprising:
an edge system, comprising:
one or more processors configured to:
connect to a cloud service;
maintain an edge index of data stored locally at the edge system;
sync, based on the edge index, at least a subset of the data comprised at the edge system with cloud data stored at the cloud service; and
provide access to the data stored locally at the edge system to one or more client systems connected to the edge system; and
a memory coupled to the one or more processors and configured to provide the one or more processors with instructions.

2. The system of claim 1, wherein the data at the edge system and the cloud data stored at the cloud service comprises patient information.

3. The system of claim 2, wherein the patient information comprises one or more of medical images, medical requisition information, or patient biographical information.

4. The system of claim 1, wherein in response to determining that a connection between the cloud service and the edge system is lost, the one or more client systems use the data stored locally at the edge system, and new data generated by the one or more client systems is stored locally at the edge system until the connection is restored between the cloud service and the edge system.

5. The system of claim 4, wherein in response to determining that the connection is restored, causing at least a subset of the new data to be synced with the cloud service.

6. The system of claim 1, wherein the edge system is an on-premises deployment of the service.

7. The system of claim 1, wherein the one or more client systems comprise an imaging system that performs a medical imaging of a patient.

8. The system of claim 1, wherein syncing the at least the subset of the data comprises:
translating the data stored locally at the edge system to translated data that is in a predefined data format associated with the cloud service; and
causing the translated data to be transferred to the cloud service.

9. The system of claim 8, wherein the data stored locally at the edge system is formatted according to a Digital Imaging and Communications in Medicine (DICOM) standard format.

10. The system of claim 1, wherein the edge system pre-fetches files that are expected to be used within a threshold period of time.

11. The system of claim 1, wherein a particular piece of data is transferred to the edge system based at least in part on schedule information that indicates when the particular piece of data is expected to be used at a client system connected to the edge system.

12. The system of claim 1, further comprising:
a cloud system to provide the cloud service to the edge system, the cloud system comprising:
one or more processors configured to:
connect to the edge system;
maintain a cloud index of the cloud data stored locally at the cloud system; and
sync, based on the cloud index of the cloud data, at least the subset of the data comprised at the edge system with the cloud data stored at the cloud system; and
a memory coupled to the one or more processors and configured to provide the one or more processors with instructions.

13. The system of claim 12, wherein syncing the at least the subset of the data comprised at the edge system with the cloud data stored at the cloud system comprises:
determining the subset of the data to be transferred between the edge system and the cloud system based at least in part on the edge index and the cloud index.

14. The system of claim 13, wherein determining the subset of the data to be transferred between the edge system and the cloud system comprises:
determining the cloud data to be transferred from the cloud system to the edge system, including:
determining a set of cloud files to be transferred to the edge system;
determining a plurality of cloud chunks corresponding to the set of cloud files to be transferred;
determining, based at least in part the edge index, a subset of cloud chunks of the plurality of cloud chunks to transfer to the edge system based on a determination of which of the plurality of cloud chunks the edge system stores copies as edge chunks; and
causing the subset of the cloud chunks to be transferred to the edge system.

15. The system of claim 14, wherein the subset of the cloud chunks is transferred based on a deduplication of the edge chunks stored at the edge system.

16. The system of claim 15, wherein after performing the deduplication, the cloud system performs a compression on a deduplicated set of cloud chunks.

17. The system of claim 15, wherein the deduplication of the edge chunks is determined based at least in part on a hash or a signature associated with each edge chunk or cloud chunk.

18. The system of claim 14, wherein the causing the subset of the cloud chunks to be transferred to the edge system comprises:
grouping the subset of the cloud chunks into a group of blocks, wherein a first block of the group of blocks comprises a plurality of chunks comprised in the subset of the cloud chunks; and
transferring the group of blocks to the edge system.

19. The system of claim 1, wherein the edge system is connected to a medical service system that stores medical data in a predefined medical data format.

20. A method, comprising:
connecting, by one or more processors associated with an edge system, to a cloud service;
maintaining an edge index of data stored locally at the edge system;
syncing, based on the edge index, at least a subset of the data comprised at the edge system with cloud data stored at the cloud service; and
providing access to the data stored locally at the edge system to one or more client systems connected to the edge system.

21. A computer program product embodied in a non-transitory computer readable medium and comprising computer instructions for:
connecting, by one or more processors associated with an edge system, to a cloud service;
maintaining an edge index of data stored locally at the edge system;
syncing, based on the edge index, at least a subset of the data comprised at the edge system with cloud data stored at the cloud service; and
providing access to the data stored locally at the edge system to one or more client systems connected to the edge system.
